(19) United States
(12) Patent Application Publication
(10) Pub. No.: US 2006/0128805 A1
(43) Pub. Date: Jun. 15, 2006

(54) Methods of treating erythropoietin-resistance

(76) Inventor: Sudhir V. Shah, Little Rock, AR (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/271,497

(22) Filed: Nov. 10, 2005

Related U.S. Application Data
(60) Provisional application No. 60/629,606, filed on Nov. 19, 2004.

(51) Int. Cl. A61K 31/195

(52) U.S. Cl. 514/564; 514/566

(57) ABSTRACT

A human having an erythropoietin resistant condition is treated by the administration of an iron chelator. The human can have end-stage renal disease, chronic renal disease, cancer or anemia. Administration of the iron chelator can essentially halt or diminish an erythropoietin resistant condition in a human.

### RELATED APPLICATION

This application claims the benefit of U.S. Provisional Application No. 60/629,606, filed Nov. 19, 2004, the entire teachings of which is incorporated hereby incorporated by reference in its entirety.

### BACKGROUND OF THE INVENTION

Erythropoiesis is the process of red blood cell (erythrocyte) formation. Red blood cells transport oxygen to tissues. The human heart and lungs function to supply continuous movement and oxygenation of red blood cells. In humans, the kidney can detect low levels of oxygen in the blood and respond by releasing the hormone erythropoietin into the bloodstream.

Renal failure can be accompanied by a state of erythropoietin deficiency. Humans with end-stage renal disease or chronic renal failure can have low plasma concentrations of erythropoietin resulting in anemia. In addition, patients suffering from conditions associated with reduced erythropoiesis, such as cancer patients, can also have reduced erythropoietin. Recombinant human erythropoietin can be administered to humans with a reduced state of erythropoiesis to stimulate erythropoiesis and, thus, the production of red blood cells. However, certain patients do not respond to erythropoietin therapy. Failure to respond to erythropoietin therapy can be associated with, for example, iron deficiency, infection, uremia, blood loss, secondary hyperparathyroidism and interactions with certain drugs (e.g., interferons, angiotenson II type I receptor blockers) (Eknoyan, G., et al., New Engl J Med 349:210-219 (2002); Ates, K., et al., Kidney Int 60:767-776 (2001); Brown, E. A., et al., J Am Soc Nephrol 14:2948-2957 (2003)). Currently there are no effective treatment options for patients that are resistant to erythropoietin treatment. Therefore, there is a need to develop new, improved and effective methods of treating patients that are resistant to erythropoietin therapy.

### SUMMARY OF THE INVENTION

The present invention relates to methods of treating erythropoietin resistance in a human.

In one embodiment, the invention is a method for treating a human, comprising the step of administering an iron chelator to a human having an erythropoietin-resistant condition.

In another embodiment, the invention is a method to essentially halt erythropoietin-resistance in a human, comprising the step of administering an iron chelator to a human that is erythropoietin-resistant.

The invention described herein provides methods for treating erythropoietin resistance. The methods of the invention can provide an effective manner to treat erythropoietin resistance and, ultimately, stimulate erythropoiesis in the human.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

In one embodiment, the invention is a method for treating a human, comprising the step of administering an iron chelator to a human having an erythropoietin-resistant condition. “Erythropoietin-resistant condition,” as used herein, means that a human with a reduced state of erythropoiesis, such as a human with end-stage renal disease, chronic renal disease, cancer or anemia, has a diminished or defective response to the administration of erythropoietin, such as recombinant erythropoietin epoetin alpha (Amgen, Corp., Thousand Oaks, Calif.). The diminished or defective response to erythropoietin results in reduced erythropoiesis, which can be assessed by decreased hematocrit and decreased hemoglobin compared to a human without an erythropoietin-resistant condition.

Erythropoietin resistance can also be assessed by the erythropoietin resistance index (ERI). ERI is calculated as the average weekly erythropoietin dose administered to the human divided by the mean hematocrit value of the human or the mean hemoglobin level of the human. Certain factors have been associated with erythropoietin resistance, including, for example, iron deficiency, infection, inflammation, blood loss, vitamin deficiency and malnutrition.

“Erythropoietin-resistant condition” is also referred to herein as “erythropoietin resistance,” “resistance to erythropoietin” or “erythropoietin resistant.” “Erythropoietin” is also referred to as “erythropoietic hormone,” “hematopoietin” and “hemopoietin.”

Generally, humans with an erythropoietin-resistant condition require a dose of erythropoietin greater than about 300 Units per kg of body weight per week (300 U/kg/wk) subcutaneously or about 450 U/kg/wk intravenously to result in an indication (e.g., increased hematocrit (Hct) level, increased hemoglobin (Hgb) level) that the erythropoietin is stimulating erythropoiesis, albeit to appreciably low levels with minimal effect on red blood cell production (NKF-K/DOQI Clinical Practice Guidelines for Anemia of Chronic Kidney Disease: Am J Kidney Dis 37:S182-238 (2001); Horl W. H., et al., Nephrol Dial Transplant 15 Suppl 4:43-50 (2000)). Generally, humans undergoing erythropoietin therapy, who are not erythropoietin resistant, require about 80-120 U/kg/wk erythropoietin (NKF-K/DOQI Clinical Practice Guidelines for Anemia of Chronic Kidney Disease: Am J Kidney Dis 37:S182-238 (2001)). Elevated doses of erythropoietin increase health care costs and may result in increased health risks, including adverse effects on blood pressure and mortality (Zhang, Y., et al., Am. J. Kidney Diseases 44: 866-876 (2004); Vaziri, N. D., Am. J. Kidney Dis. 33:821-828 (1999)).

Erythropoiesis, the process of forming new red blood cells, requires iron and erythropoietin. Erythropoietin is secreted by the kidney in response to low oxygen levels in the human and iron is an energy source for producing the red blood cells. In humans who are deficient in iron are anemic because iron is not available for use by blood marrow in response to erythropoietin.

Humans with anemia of chronic disease, such as hemolytic anemia, idiopathic aplastic anemia, idiopathic autoimmune hemolytic anemia or immune hemolytic anemia, are unable to effectively mobilize iron from iron stores for use in erythropoiesis (Jurado R. L., Clin Infect Dis 25: 888-895 (1997); Jongen-Lavrencic M., et al., Clin Exp Immunol 103: 328-334 (1996); van Gameren M. M., et al., Blood 84:1434-1441 (1994); Schooley J. C., et al., Br J Haematol 67:11-17 (1987)). Administration of deferiprone, an iron chelator, can increase hemoglobin, a clinical marker of erythropoiesis (Vreugdenhil G., et al., Lancet 2:1398-1399 (1989); al-Refaie F. N., et al., Recent Adv Hematol 7:185-216 (1993)). Thus, it is believed that the administration of an iron chelator to a human having an erythropoietin resistant condition will mobilize iron from iron stores (e.g., from the reticulo-endothelial system) for use in erythropoiesis in response to erythropoietin administration to the human, to thereby treat the erythropoietin resistant condition in the human and increase red blood cells in the human.

A human having an erythropoietin-resistant condition can have end-stage renal disease. End-stage renal disease (ESRD), also referred to as end-stage kidney disease, is a complete or near complete failure of the kidneys to function to excrete wastes, concentrate urine and regulate electrolytes. ESRD occurs when the kidneys are no longer able to function at a level that is necessary for day-to-day life. Humans with ESRD (or chronic kidney disease, anemia or cancer, see below) can be erythropoietin deficient due to the failure of the kidneys to detect low oxygen and release erythropoietin. In addition, humans with ESRD, who are erythropoietin resistant, can have high levels iron stores because the iron is not being utilized in erythropoiesis. Iron overload can result in serious health risks, including cardiovascular disease and early mortality.

ESRD generally occurs as chronic renal failure progresses to the point where kidney function is less than about 10% of a normal, disease-free kidney. In ESRD, kidney function is so low that without dialysis or kidney transplantation, complications are multiple and severe resulting in death from an accumulation of fluids and waste products in the body.

The most common cause of ESRD is diabetes. ESRD generally follows chronic kidney failure, which may exist for 2 to 20 years or more before progression to ESRD. Symptoms of ESRD can include, for example, unintentional weight loss, nausea or vomiting, general ill feeling, fatigue, headache, decreased urine output, easy bruising or bleeding, blood in the vomit or stools, elevated creatinine and blood urea nitrogen (BUN) levels and decreased creatinine clearance.

Renal dialysis (hemodialysis) or kidney transplantation are treatments for ESRD. A human with ESRD treated by the methods described herein can be undergoing renal dialysis or have undergone a kidney transplant. The iron chelator can be administered to the human before renal dialysis or kidney transplant, during renal dialysis or kidney transplant and after renal dialysis or kidney transplant.

Complications of ESRD include, for example, anemia, pericarditis, atherosclerosis and consequences such as myocardial infarction, cardiac tamponade, congestive heart failure left ventricular hypertrophy, hypertension, platelet dysfunction, gastrointestinal loss of blood, duodenal or peptic ulcers, hemorrhage, infection and mortality (Silverberg, D., et al., Curr Opin Nephrol Hypertens 13:163-170 (2004); Hampl H., et al., Clin Nephrol 58 Suppl 1:S73-96 (2002); Levin A., et al., Am J Kidney Dis 36:S24-30 (2000); Foley, R. N., et al., Am J Kidney Dis 28:53-61 (1996); Ma, J. Z., et al., J Am Soc Nephrol 10:610-619 (1999)). The methods of the invention described herein may prevent or alleviate complications of ESRD by alleviating the resistance to erythropoietin and mobilizing iron stores in the human to thereby stimulate erythropoiesis.

Humans on hemodialysis can have a poor response to erythropoietin despite having adequate iron stores. Adverse consequences may occur in these erythropoietin-resistant patients including failure to achieve the recommended hemoglobin of about 11 grams/dL to about 12 grams/dL and/or a hematocrit of about 33% to about 36% of the total blood, anemia, and the likelihood of requiring higher doses of iron, which has been associated with inflammation and oxidative stress, as well as increased carotid intima-medial thickness.

A human having an erythropoietin-resistant condition can have chronic kidney disease (CKD). Chronic kidney disease is a gradual and progressive loss of the ability of the kidneys to excrete waste, concentrate urine and conserve electrolytes. Chronic kidney disease is also referred to as chronic renal insufficiency and chronic kidney failure. Unlike acute renal failure with its sudden reversible failure of kidney function, chronic renal failure is slowly progressive. Chronic kidney disease can result from any disease that causes gradual loss of kidney function and can range from mild dysfunction of the kidney to severe kidney failure. Progression of the chronic kidney disease can continue to ESRD. Chronic kidney disease usually occurs over a number of years as the internal structures of the kidney are slowly damaged. Clinical parameters indicative of chronic kidney disease include an increase in creatinine levels, an increase in blood urea nitrogen (BUN) levels, a decrease in creatinine clearance and a decrease in glomerular filtration rate (GFR).

Diseases that cause chronic renal failure can include, for example, diabetes mellitus, glomerulonephritis, hypertension, obstruction to the urinary tract and polycystic kidney disease. Complications or consequences of chronic kidney disease include, for example, anemia, hypertension, congestive heart failure and inflammation. The methods of the invention described herein may reduce the severity or alleviate such complications or consequences by alleviating the resistance to erythropoietin in the human with chronic kidney disease to thereby stimulate erythropoiesis.

A human is also referred to herein as a patient or a subject.

In another embodiment, the human with an erythropoietin-resistant condition has cancer, such as an epithelial cancer (e.g., breast cancer, skin cancer), bone marrow cancer, connective tissue cancer, nervous system cancer. Cancer can result in reduced erythropoiesis, for example, as a consequence of depletion of the red bone marrow by chemotherapy or radiation treatment. A human that has cancer and is erythropoietin-resistant can be undergoing cancer treatment, such as a chemotherapy treatment, radiation treatment, stem cell replacement treatment.

The human having an erythropoietin-resistant condition treated by the methods of the invention can be anemic. The anemia can be associated with ESRD, chronic kidney disease, cancer, anemia of unknown origin or anemia due to chronic disease in the human. Anemia is a common complication of chronic kidney disease and ESRD, particularly in humans undergoing hemodialysis as a renal replacement therapy (Zhang, Y., et al., Am. J. Kidney Diseases 44: 866-876 (2004)).

The severity of anemia in the human having an erythropoietin resistant condition can be essentially halted, diminished or normalized following the administration of the iron chelator to the human. “Essentially halted,” as used herein in reference to anemia, refers to failure of the anemia to progress in the human over time (e.g., days, weeks, months, or years). “Diminished,” as used herein in reference to anemia, means that the anemia is reduced in the human following administration of the iron chelator. Blood parameters to measure anemia and halting of anemia in the human include, for example, hemoglobin levels and hematocrit levels before and after treatment with the iron chelator. Hemoglobin and/or hematocrit levels of about those observed in humans without erythropoietin resistance (about 11 g/dL to about 12 g/dL; and about 33% to about 36%, respectively, or lower than those observed in the human prior to treatment) following administration of an iron chelator to a human with erythropoietin resistant condition indicates that the anemia is essentially halted, diminished or normalized.

A receiver operative characteristic (ROC) can be used to assess severe anemia, (hemoglobin<10 gm/dL) (Greenwood R. N., et. al., Kidney Int Suppl: S78-86 (2003.)) In a recent ESRD Clinical Performance Measures Project (DHHS: 2003 Annual Report: Am J Kidney Dis 44:S28-S32 (2004)), about 20% of ESRD patients had hemoglobin<11 gm/dL and are, thus, were erythropoietin resistant.

In anemia of chronic disease, there is an inability to mobilize iron from iron stores for erythropoiesis. The iron chelator deferiprone can mobilize iron from iron stores (al-Refaie F. N., et al., Recent Adv Hematol 7:185-216 (1993); Vreugdenhil G, et. al., Ann Rheum Dis 49:956-957 (1990)), including the storage iron proteins ferritin and hemosiderin (Kontoghiorghes G. J., et al., Biochem J 241:87-92 (1987)) and unlike the iron chelator deferoxamine, from iron-saturated transferrin and lactoferrin (Kontoghiorghes G. J., Biochemica et Biophysica Acta 882.267-270 (1986)). The mobilization of iron from ferritin and hemosiderin is gradual, which can take days (Kontoghiorghes G. J., Inorg Chim Acta 138 (1987)), whereas mobilization of iron from transferrin and lactoferritin is completed within hours (Kontoghiorghes G. J., Biochemica et Biophysica Acta 882:267-270 (1986); Kontoghiorghes G. J., Biochim Biophys Acta 869:141-146 (1986)).

Deferiprone can mobilize iron from both reticuloendothelial and hepatocellular pools (Barman Balfour J. A, et al., ADIS Drug Evaluation 58:553-578 (1999)). It is believed that following the administration of an iron chelator humans with a erythropoietin-resistant condition can mobilize iron stores for use in erythropoiesis resulting in an increased red blood cell count.

A human having an erythropoietin-resistant condition treated by the methods of the invention can have inflammation. Inflammation generally is a localized protective response elicited by injury or destruction of tissues that destroys, dilutes or sequesters the injurious agent and injured tissue. Erythropoietin resistance is frequently associated with inflammatory conditions. The inflammatory process may be acute, resulting in a transient resistance to erythropoietin, or chronic, with a persistently poor response to erythropoietin. Administration of an iron chelator to a human having erythropoietin resistance can reduce the inflammation.

The human having erythropoietin resistance can have a systemic inflammation or an acute phase response to inflammation. Systemic inflammation is inflammation throughout the body.

The human having erythropoietin resistance can have an acute phase response to inflammation. An acute-phase response to inflammation can be assessed by, for example, an increase in c-reactive protein (CRP), interleukin-6, tumor necrosis factor-α, amyloid A, ferritin, fibrinogen, α1-antitrgpsin and haptoglobin (referred to as positive acute-phase reactants) in a blood sample of the human. The increase in the concentration of the positive acute-phase reactants is based on the concentration of positive acute phase reactants in a human appropriately matched for gender, health and other appropriate variables, that does not have an acute-phase response (Kalantar-Zadeh, K., et al., Am. J. Kidney Diseases 42: 864-881 (2003)). The synthesis of other proteins (e.g., albumin, transferrin, prealbumin, cholesterol, leptin, histidine), referred to as negative acute-phase reactants, is decreased in the acute-phase response to inflammation (Kalantar-Zadeh, K., et al., Am. J. Kidney Diseases 42: 864-881 (2003)).

Humans on renal dialysis who are erythropoietin resistant can have increased oxidative stress and inflammation. The administration of an iron chelator to a human with erythropoietin resistance may reduce or normalize inflammation and oxidative stress and mobilize iron for erythropoiesis. “Normalize,” as used herein in reference to anemia, inflammation, or oxidative stress, means a change that approaches a value observed in a human without erythropoietin resistance. A reduction in inflammation can be assessed, for example, by a reduction in the amount of CRP, interleukin-6, tumor necrosis factor-α, amyloid A, ferritin, fibrinogen, α1-antitrgpsin and haptoglobin in a blood sample obtained from the human treated by the methods described herein in comparison to a blood sample obtained from the human prior to treatment by the methods of the invention described herein.

Humans with erythropoietin resistance can, for example, fail to achieve the recommended hematocrit and thus be anemic (Silverberg D., et al., Curr Opin Nephrol Hypertens 13:163-170 (2004); Hampl H., et al., Clin Nephrol 58 Suppl 1:S73-96 (2002); Levin A, et al., Am J Kidney Dis 3:S24-30 (2000); O'Riordan E., et al., Nephrol Dial Transplant 15 Suppl 3:19-22 (2000); NKF-K/DOQI Clinical Practice Guidelines for Anemia of Chronic Kidney Disease: Am J Kidney Dis 37:S182-238 (2001); Horl W. H., et al., Nephrol Dial Transplant 15 Suppl 4:43-50 (2000)). Anemia can require that high doses of iron be administered to the human, which has been associated with inflammation and oxidative stress (Salahudeen A. K., et al., Kidney Int 60:1525-1531 (2001); Agarwal R., et al., Semin Nephrol 22:479-487 (2002); Parkkinen J., et al., Nephrol Dial Transplant 15:1827-1834 (2000); Alam M. G., et al., Kidney Int 66: 457-458 (2004)) and can have increased carotid intima-media thickness (Drueke T., et al., Circulation 106: 2212-2217 (2002); Himmelfarb J., et al., Kidney Int 62: 1524-1538 (2002)). Administration of iron is associated with an increase in cardiac deaths in ESRD patients (Besarab A., et al., N Engl J Med 339: 584-590 (1998); Besarab A., et al., J Am Soc Nephrol 10: 2029-2043 (1999)).

Humans with CKD can also have increased levels of inflammatory and oxidative markers (Handelman G. J., et al., Kidney Int 59: 1960-1966 (2001); Nguyen-Khoa T., et al., Nephrol Dial Transplant 16:335-340 (2001); Oberg B. P., et al., Kidney Int 65: 1009-1016 (2004)). This state of inflammation has been implicated in several complications of uremia (Mezzano D., et al., Kidney Int 60:1844-1850 (2001); Sarnak M. J., et al., Kidney Int 62:2208-2215 (2002) including cardiovascular events (Owen, W. F., et al., Kidney Int 54:627-636 (1998); Kitiyakara C., et al., Curr Opin Nephrol Hypertens 9:477-487 (2000); Massy, S. A., et al., Nephrol Dial Transplant 18:153-157 (2003); Bologa R. M., et al., Am J Kidney Dis 32:107-114 (1998); Yeun, J. Y., et al., Am J Kidney Dis 35:469-476 (2000); Stenvinkel P., et al., Kidney Int 55:1899-1911 (1999)) and erythropoietin resistance (Kalantar-Zadeh K., et al., Am J Kidney Dis 42:761-773 (2003); Macdougall I. C., et al., Nephrol Dial Transplant 17 Suppl 11:39-43 (2002); Stenvinkel P., et al., Nephrol Dial Transplant 17 Suppl 5:32-37 (2002); Gunnell J., et al., Am J Kidney Dis 33:63-72 (1999); Goicoechea M., et al., Kidney Int 54:1337-1343 (1998). Macdougall I. C., et al., Nephrol Dial Transplant 17 Suppl 1: 48-52 (2002); Kato A., et al., Nephrol Dial Transplant 16: 1838-1844 (2001); Barany P., Nephrol Dial Transplant 16: 224-227 (2001); Barany P., et al., Am J Kidney Dis 29:565-568 (1997); Kalantar-Zadeh K., et al., Adv Ren Replace Ther 10: 155-169 (2003)). The administration of an iron chelator may result in reduced oxidative stress and inflammation in a human who has an erythropoietin resistant condition.

Anemia may result from inflammation. Anemia is observed frequently in humans suffering from chronic inflammatory disorders even with a normal kidney function (Voulgari P. V., et al., Clin Immunol 92: 153-160 (1999)). Inflammation is associated with iron deficiency, low levels of serum iron and transferrin, and increased serum ferritin levels. The delivery of serum iron from reticuloendothelial cells to hemopoietic cells is inhibited or blocked in humans suffering from such chronic inflammatory disorders. Serum ferritin, which is also an acute-phase reactant, increases two- to four-fold in response to inflammation. Cytokines such as TNF-α and IL-1, also directly and indirectly (by increasing iron uptake in hepatocytes) stimulate ferritin synthesis, which increases iron-storage pools.

As discussed above, humans with kidney disease have increased oxidative stress and inflammation. Iron plays an important role in generation of additional and more reactive oxidants, including the highly-reactive hydroxyl radical (or a related highly-oxidizing species) in a reaction commonly referred to as the metal-catalyzed Haber-Weiss reaction (Halliwell B., et al., Meth Enzymol 186:1-85 (1990)).
Fe³⁺+O₂⁻→Fe²⁺+O₂
Fe²⁺+H₂O₂→Fe³⁺+OH+OH.
O₂⁻+H₂O₂→O₂+OH+OH.

Iron also plays a major role in initiation and propagation of lipid peroxidation, either by catalyzing the conversion of primary oxygen radicals to hydroxyl radicals or by forming a perferryl ion. In addition, iron can directly catalyze lipid peroxidation, the oxidative reaction of polyunsaturated lipids, by removing hydrogen atoms from the polyunsaturated fatty acids in the lipid bilayers of organelle membranes. Iron is essential to the formation of mature red blood cells in the process of erythropoiesis.

The use of iron chelators can lead to a marked reduction in oxidative stress and inflammation (Matthews A. J., et al. J Surg Res 73: 35-40 (1997); Duffy S. J., et al., Circulation 103:2799-2804 (2001); Voest E. E., et al., Ann Intern Med 120: 490-499 (1994). Inflammation has been linked to erythropoietin resistance. By reducing inflammation, the iron chelator would provide benefit in treating erythropoietin resistance.

Reduction of inflammation may provide other beneficial effects. Inflammation, including humans without erythropoietin resistance, has been associated with high cardiovascular disease events (Voest E. E., et al., Ann Intern Med 120:490-499 (1994); Ross R., et al., Am J Nephrol 21:176-178 (2001)). Low-grade inflammation in patients with ESRD, characterized by increased CRP, TNF-α, IL-1 and IL-6 levels, has been associated with decreased patient survival (Bologa R. M., et al., Am J Kidney Dis 32:107-114 (1998); Yeun J. Y., et al., Am J Kidney Dis 35:469-476 (2000)), increased carotid intima-media thickness, atherosclerosis (Kato A., et al., Am J Nephrol 21: 176-178 (2001); Kato A., et al., Kidney Int 61: 1143-1152 (2002); Stenvinkel P., et al., Am J Kidney Dis 39:274-282 (2002), and poor nutrition (Kalantar-Zadeh K., et al., Am J Kidney Dis 42:761-773 (2003); Kalantar-Zadeh K., et al., Adv Ren Replace Ther 10:155-169 (2003); Ikizler T. A., et al., Kidney Int 55:1945-1951 (1999)). Inflammation can be assessed following the administration of iron chelators as well as uremia and cardiovascular disease.

Iron chelators have been used in dialysis patients (Abreo K., Sem Dialysis 1:55-61 (1988); Altman P., et al., Lancet 1:1012-1015 (1988)). Before the use of erythropoietin, the iron chelator deferoxamine was used to chelate iron in hemodialysis patients with iron overload due to blood transfusions (Stivelman J., et al., Kidney Int 36:1125-1132 (1989); Hakim R. M., et al., Am J Kidney Dis 10:293-199 (1987)). Erythropoietin was also used to chelate aluminum, which was frequently used in the past for hyper-phospotemia (Altman P., et al., Lancet 1:1012-1015 (1988); Abreo K., Sem Dialy 1:55-61 (1988)). Deferiprone has been used to treat aluminum overload and iron overload. A mean increase in plasma aluminum of about 90% within the first hour of oral administration of deferiprone has been observed, indicating the ability of deferiprone to mobilize aluminum (Kontoghiorghes G. J., et al., Drug Safety 26:553-584 (2003). In addition, deferiprone iron complex is dialyzable, indicating that this complex or deferiprone are unlikely to accumulate in hemodialysis patients (Kontoghiorghes G. J., et al., Drug Safety 26:553-584 (2003). Side effects of deferiprone are known (Kontoghiorghes G. J., et al., Drug Safety 26:553-584 (2003).

The mean change in epoetin resistance index (epoetin dose per wk/Hgb) will be assessed following administration of the iron chelator. The epoetin resistance index was described by Gunnell et al (Gunnell J., et al., Am J Kidney Dis 33:63-72 (1999)) and is a well accepted assessment of epoetin resistance. The epoetin resistance has two components (variables): epoetin dose and hemoglobin/hematocrit levels. An intervention leading to modification in epoetin resistance can lead to either change in epoetin dose or change in Hgb/Htc levels or both. As an outcome variable just one variable (epoetin dosage or Htc) alone cannot be used to evaluate the effect of an intervention. Gunnell used a ratio of the dosage of erythropoietin to percent Htc (e.g., epoetin/Htc) to assess epoetin resistance, referred to as epo-responsiveness index or erythropoietin resistance index.

EPI can be normalized for body weight [epo/(weight×Hgb)], hemoglobin, intravenous iron requirement, and epoetin dose. Markers of inflammation such as CRP, TNF-α, IL-1, and IL-6 can also be measured following administration of the iron chelator. Isoprostane levels can be measured as a marker for oxidative stress. Isoprostanes, which result primarily from the non-enzymatic alteration of arachidonic acid by reactive oxidant species, have emerged as important biomarkers of oxidative stress in vivo (Roberts L. J. I., et al., Cell Mol Life Sci 59:808-820 (2002); Morrow J. D., Drug Metab Rev 32:377-385 (2000) (for review see reference (Roberts L. J. I., et al., Cell Mol Life Sci 59:808-820 (2002)). 8-isoprostane (8-epi PGF2a) has been widely used in recent studies as a marker of oxidative stress (Forgione M. A., et al., Circulation 106:1154-1158 (2002); Levonen A. L., et al., Biochem J 378:373-382 (2004); Ishizuka T., et al., J Cardiovasc Phar-macol 141:571-578 (2003); Brault S., et al., Stroke 34:776-782 (2003)).

An “iron chelator” refers to any molecule capable of interacting with iron, either Fe³⁺ or Fe²⁺, to prevent the formation of catalytic iron from Fe³⁺ or to prevent, inhibit or interfere with iron (Fe³⁺ or Fe²⁺) interacting, effecting or participating in the Haber-Weiss reaction (supra) or any other reaction which can generate hydroxyl radicals. The interaction between the iron chelator and iron, either Fe³⁺, Fe²⁺, or both, can be, for example, a binding interaction, an interaction as a result of steric hindrance or any reciprocal effect between iron and the iron chelator. The iron chelator can, for example, prevent the conversion of Fe³⁺ to Fe²⁺, thereby indirectly preventing the reduction of hydrogen peroxide and formation of hydroxyl radicals in the Haber-Weiss reaction. Alternatively, or additionally, the iron chelator can interact directly with Fe²⁺ to prevent hydroxyl radical formation in, for example, the Haber-Weiss reaction.

The iron chelator can be a peptide comprising natural or nonnatural (e.g., amino acids not found in nature) amino acids, polyethylene glycol carbamates, lipophilic or nonlipophilic polyaminocarboxylic acids, polyanionic amines or substituted polyaza compounds. The iron chelator can be at least one member selected from the group consisting of deferiprone (1,2-dimethyl-3-hydroxy-pyrid-4-one)L1, desferrithiocon, hydroxybenzyl-ethylenediamine-diacetic acid and pyridoxal isonicotinoyl hydrazone. The iron chelator can be at least one member selected from the group consisting of 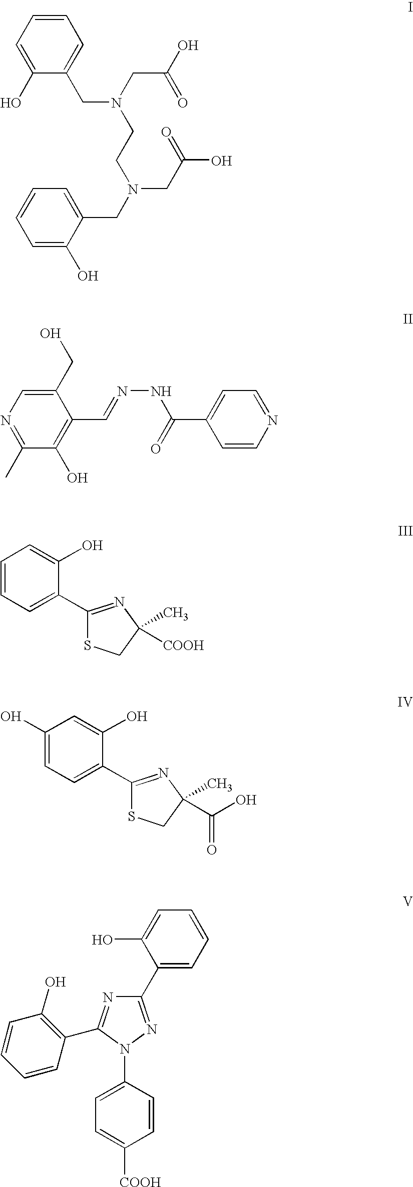
Iron chelators are commercially available or can be synthesized or purified from biological sources using routine procedures.

Exemplary descriptions and discussions of iron chelators are found in several references, for example, U.S. Pat. No. 5,047,421 (1991); U.S. Pat. No. 5,424,057 (1995); U.S. Pat. No. 5,721,209 (1998); U.S. Pat. No. 5,811,127 (1998); Olivieri, N. F. et al., New Eng J. Med. 332:918-922 (1995); Boyce, N. W. et al., Kidney International. 30:813-817 (1986); Kontoghiorghes, G. J. Indian J. Peditr. 60:485-507 (1993); Hershko, C. et al., Brit. J Haematology 101:399-406 (1998); Lowther, N. et al., Pharmac. Res. 16:434 (1999); Cohen, A. R., et al., Am. Soc. Hematology pages 14-34 (2004)); U.S. Pat. No. 6,993,104 (2005); U.S. Pat. No. 6,908,733 (2005); U.S. Pat. No. 6,906,052 (2005), the teachings of all of which are hereby incorporated by reference in their entirety.

An “amount effective,” when referring to the amount of iron chelator is defined as that amount (also referred to herein as dose) of iron chelator that, when administered to a human having erythropoietin resistance, is sufficient for therapeutic efficacy (e.g., an amount sufficient to reduce, or eliminate, the erythropoietin resistance or to stimulate erythropoiesis).

In one embodiment, the iron chelator is administered in a single dose. In another embodiment, the iron chelator is administered in multiple doses. The iron chelator can be administered orally at a dose of between about 10 mg/kg and about 150 mg/kg of the human and between about 20 mg/kg body of the human and about 150 mg/kg body weight of the human. In a particular embodiment, the iron chelator is administered three times a day at a dose in a range of between about 20 mg/kg body of the human and about 150 mg/kg body weight of the human.

Failure to respond to erythropoietin administration in humans with reduced erythropoiesis results in decreased red blood cells, decreased hematocrit and decreased hemoglobin. Humans with erythropoietin resistance, are frequently administered a source of iron, such as iron gluconate and iron sucrose. In another embodiment, the methods of the invention further includes the step of administering iron (e.g., iron gluconate, iron sucrose) to the human having an erythropoietin resistant condition. The iron chelator would not be administered to the human the day the human is being treated with an iron. The amount of iron in a blood sample of the human can be measured and the dose of the iron chelator and exogenous source of iron adjusted, as needed. One of skill in the art would be capable of adjusting the dose of iron and iron chelator to administered to the human to achieve a sufficient amount of iron available for use in erythropoiesis in the human.

The method of treating a human having an erythropoietin resistant condition with an iron chelator can further include the step of administering an erythropoiesis regulator to the human. “Erythropoiesis regulator,” as used herein, refers to a compound (e.g., protein, peptide) that alters erythropoiesis in the human. The erythropoiesis regulator can stimulate erythropoiesis in the human. The erythropoiesis regulator can decrease or inhibit erythropoiesis in the human. The erythropoiesis regulator can be administered to the human before, during (also referred to as concomitantly) and after administration of the iron chelator. One of skill in the art would be able to adjust the dose of an erythropoiesis regulator that would be administered to the human.

In a particular embodiment, recombinant human erythropoietin (epoetin alpha; EPOGEN®; Amgen, Inc., Thousand Oaks, Calif.) is administered to the human having an erythropoietin resistant condition who is being treated by the methods of the invention.

The dose of an erythropoiesis regulator administered to the human following administration of the iron chelator can be less than the dose of the erythropoiesis regulator administered to the human prior to administration of the iron chelator. As discussed above, it is believed that administration of an iron chelator to a human having an erythropoietin-resistant condition, will chelator iron stores from, for example, the reticuloendothelial system for use in erythropoiesis. As also discussed above, increased doses of erythropoietin exceeding about 300 U/kg/wk can have negative side effects and, thus, can be undesirable. Thus, treatment of a human having an erythropoietin resistant condition can eliminate the need to increase the dose of erythropoietin resulting in doses of erythropoietin that are within a range generally administered to humans who are not resistant to erythropoietin (e.g., between about 80 U/kg/wk to about 120 U/kg/wk).

As discussed above, erythropoietin resistance can be measured by calculating the ERI. The ERI can be calculated before during and after the administration of an iron chelator to a human having an erythropoietin resistant condition. Thus, the methods of the invention described herein can further include the step of measuring the dose of an erythropoiesis regulator relative to a change in at least one member selected from the group consisting of a hemoglobin level and a hematocrit level in the human.

The methods of the invention can further include the step of measuring iron content in a blood sample obtained from the human; the step of measuring a ferritin reticulocyte count in a blood sample obtained from the human; and the step of measuring total iron binding capacity (TIBC) in a blood sample obtained from the human.

Additionally, or alternatively, the methods of the invention can further include the step of measuring the complete blood count in a blood sample obtained from the human. In particular, the hemoglobin content and hematocrit can be determined in the complete blood count. An increase in iron, ferritin reticulocyte count, total iron binding capacity, hemoglobin content and hematocrit can indicate the availability of iron for use in erythropoiesis and, thus, increased stimulation in erythropoiesis to thereby increase red blood cells.

In an additional embodiment, the invention is a method to essentially halt or diminish, erythropoietin-resistance in a human, comprising the step of administering an iron chelator to a human that is erythropoietin-resistant. “Essentially halt,” as used herein when referring to erythropoietin resistance in a human, refers to preventing, either temporarily or permanently, the resistance to erythropoietin in the human. “Diminish,” as used herein in reference to erythropoietin-resistance in a human, means that the human can respond to erythropoietin administration, which can be manifested by stimulation of erythropoiesis. When the erythropoietin-resistance is diminished, the human can be administered a lower dose of erythropoietin.

Essentially halting or diminishing the erythropoietin resistance can be assessed by, for example, increasing the available stores of iron for erythropoiesis that can in turn be assessed by, for example, an increase in hematocrit, an increase in hemoglobin, a decrease in the dose of erythropoietin administered to the human (e.g., a decrease from about 300 U/kg to about 200 U/kg), a decrease in ERI or a decrease in serum iron levels.

The methods of the present invention can be accomplished by the administration of the iron chelator iron by enteral or parenteral means. Specifically, the route of administration is by oral ingestion (e.g., tablet, capsule form, pill). Other routes of administration as also encompassed by the present invention including intramuscular, intravenous, intraarterial, intraperitoneal, or subcutaneous routes, nasal administration, suppositories and transdermal patches.

The iron chelators can be used alone or in any combination when administered to the humans. For example, deferiprone can be coadministered with another iron chelator such as deferoxiamine to treat an erythropoietin resistance (e.g., in a human with chronic renal disease, end-stage renal disease or cancer). It is also envisioned that one or more iron chelators can be coadministered with other therapeutics (e.g., erythropoietin) to, for example, to treat the human (e.g., to stimulate erythropoiesis). Coadministration is meant to include simultaneous or sequential administration of two or more iron chelators. It is also envisioned that multiple routes of administration (e.g., intramuscular, oral, transdermal) can be used to administer one or more iron chelators.

The iron chelators can be administered alone or as admixtures with conventional excipients, for example, pharmaceutically, or physiologically, acceptable organic, or inorganic carrier substances suitable for enteral or parenteral application which do not deleteriously react with the iron chelator. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the iron chelator.

When parenteral application is needed or desired, particularly suitable admixtures for the iron chelator are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The iron chelators can also be administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of both of which are hereby incorporated by reference.

The dosage and frequency (single or multiple doses) of iron chelators administered to a human can vary depending upon a variety of factors, including the size, age, sex, health, body weight, body mass index, and diet of the human; nature and extent of symptoms of the erythropoietin resistance or other condition of the human (e.g., chronic renal disease, end-stage renal disease, cancer), kind of concurrent treatment (e.g., erythropoietin, dialysis, chemotherapy, radiation therapy), complications from the a condition that the human has (e.g., chronic renal disease, end-stage renal disease, cancer) or other health-related problems. In a preferred embodiment, humans with a kidney disease are treated three times a day with a dose of iron chelator (e.g., deferiprone in 500 mg capsules) at about 30 mg/kg to about 75 mg/kg body weight per day for about 2-6 months. Other therapeutic regimens or agents can be used in conjunction with the iron chelator treatment methods of the present invention. For example, the administration of the iron chelator can be accompanied by erythropoietin administration, chemotherapy or radiation therapy. Adjustment and manipulation of established dosages (e.g., frequency and duration) are well within the ability of those skilled in the art.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for treating a human, comprising the step of administering an iron chelator to a human having an erythropoietin-resistant condition.

2. The method of claim 1, wherein the human has end-stage renal disease.

3. The method of claim 1, wherein the human has chronic kidney disease.

4. The method of claim 2, wherein the human is undergoing renal dialysis.

5. The method of claim 4, wherein the iron chelator is administered before renal dialysis and after renal dialysis.

6. The method of claim 1, wherein the human as cancer.

7. The method of claim 6, wherein the human is undergoing a cancer treatment.

8. The method of claim 7, wherein the cancer treatment is a chemotherapy treatment.

9. The method of claim 7, wherein the cancer treatment is a radiation treatment.

10. The method of claim 1, wherein the human is anemic.

11. The method of claim 1, wherein the human has inflammation.

12. The method of claim 11, wherein the iron chelator is administered in an amount sufficient to reduce inflammation in the human following the administration of the iron chelator to the human.

13. The method of claim 11, wherein the inflammation is a systemic inflammation.

14. The method of claim 11, wherein the inflammation is an acute phase response to inflammation.

15. The method of claim 14, further including measuring in a blood sample obtained from the human at least one member selected from the group consisting of c-reactive protein, interleukin-6, tumor necrosis factor-α, amyloid A, ferritin, fibrinogen, α1-antitrgpsin and haptoglobin.

16. The method of claim 1, wherein the iron chelator is administered at a dose of between about 10 mg/kg and about 150 mg/kg.

17. The method of claim 1, wherein the iron chelator is administered in a single dose.

18. The method of claim 1, wherein the iron chelator is administered in multiple doses.

19. The method of claim 1, wherein the iron chelator is administered orally.

20. The method of claim 1, wherein the iron chelator is at least one member selected from the group consisting of deferiprone, deferoxamine, polyanionic amines, substituted polyaza compounds, desferrithiocon, hydroxybenzyl-ethylenediamine-diacetic acid and pyridoxal isonicotinoyl hydrazone.

21. The method of claim 1, wherein the iron chelator is at least one member selected from the group consisting of:

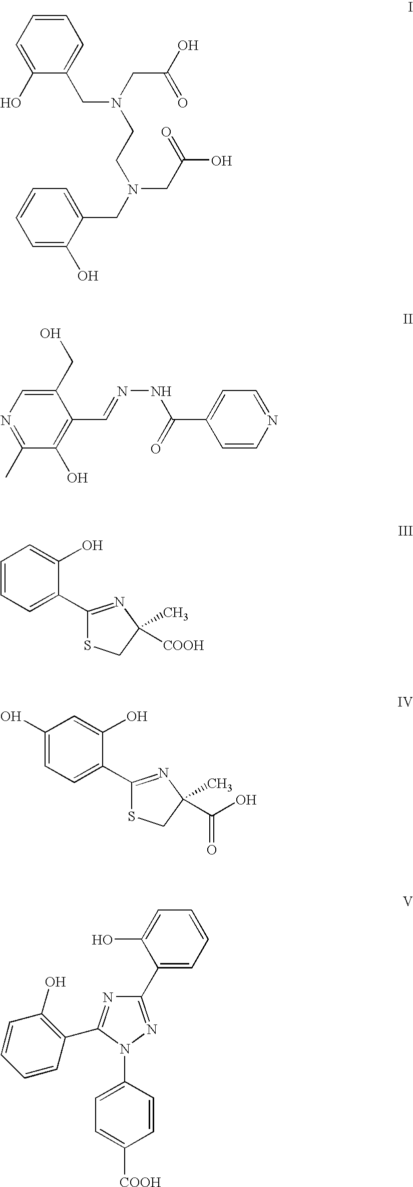

22. The method of claim 10, wherein the severity of anemia in the human is essentially halted following the administration of the iron chelator to the human.

23. The method of claim 1, further including the step of administering iron to the human.

24. The method of claim 23, wherein the iron administered to the human is in the form of at least one member selected from the group consisting of iron gluconate and iron sucrose is administered to the human.

25. The method of claim 1, further including the step of measuring iron in a blood sample obtained from the human.

26. The method of claim 1, further including the step of administering an erythropoiesis regulator to the human.

27. The method of claim 26, wherein the erythropoiesis regulator stimulates erythropoiesis in the human.

28. The method of claim 27, wherein the erythropoiesis regulator is erythropoietin.

29. The method of claim 27, wherein the dose of the erythropoiesis regulator administered to the human following administration of the iron chelator is less than the dose of the erythropoiesis regulator administered to the human prior to administration of the iron chelator.

30. The method of claim 1, further including the step of measuring the dose of an erythropoiesis regulator relative to a change in at least one member selected from the group consisting of a hemoglobin level and a hematocrit level in the human.

31. The method of claim 1, further including the step of measuring iron content in a blood sample obtained from the human.

32. The method of claim 1, further including the step of measuring a ferritin reticulocyte count in a blood sample obtained from the human.

33. The method of claim 1, further including the step of measuring the complete blood count in a blood sample obtained from the human.

34. The method of claim 1, further including the step of measuring total iron binding capacity in a blood sample obtained from the human.

35. A method to essentially halt erythropoietin-resistance in a human, comprising the step of administering an iron chelator to a human that is erythropoietin-resistant.
